SEQUENCE LISTING (68 sequences)

SEQ ID NO: 1 - DNA, 1541 bp, Escherichia coli
aaattgaagagtttgatcatggctcagattgaacgctggcggcaggcctaacacatgcaagtcgaacggtaacaggaagaagcttgctctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 2 - DNA, 19 bp, Escherichia coli
gagtttgatcctggctcag

SEQ ID NO: 3 - DNA, 20 bp, Artificial
  Synthetic Primer
gtttgatcmtggctcaggac

SEQ ID NO: 4 - DNA, 20 bp, Artificial
  Synthetic Primer
gtttgatcmtggctcagrac

SEQ ID NO: 5 - DNA, 18 bp, Artificial
  Synthetic Primer
ttgatcmtggctcagrac

SEQ ID NO: 6 - DNA, 22 bp, Artificial
  Synthetic Primer
gagtttgatcmtggctcagakt

SEQ ID NO: 7 - DNA, 21 bp, Artificial
  Synthetic Primer
gagtttgatcmtggctcagak

SEQ ID NO: 8 - DNA, 22 bp, Artificial
  Synthetic Primer
gagtttgatcmtggctcagakt

SEQ ID NO: 9 - DNA, 21 bp, Artificial
  Synthetic Primer
gtttgatcmtggctcagaktg

SEQ ID NO: 10 - DNA, 20 bp, Artificial
  Synthetic Primer
gtttgatcmtggctcagakt

SEQ ID NO: 11 - DNA, 20 bp, Artificial
  Synthetic Primer
gtttgatcmtggctcagakt

SEQ ID NO: 12 - DNA, 17 bp, Artificial
  Synthetic Primer
ttgatcmtggctcagak

SEQ ID NO: 13 - DNA, 18 bp, Artificial
  Synthetic Primer
ttgatcmtggctcagakt

SEQ ID NO: 14 - DNA, 21 bp, Artificial
  Synthetic Primer
agtttgatcmtggctcaggat

SEQ ID NO: 15 - DNA, 20 bp, Artificial
  Synthetic Primer
gtttgatcmtggctcaggat

SEQ ID NO: 16 - DNA, 20 bp, Artificial
  Synthetic Primer
gtttgatcmtggctcaggat

SEQ ID NO: 17 - DNA, 18 bp, Artificial
  Synthetic Primer
gtattaccgcggctgctg

SEQ ID NO: 18 - DNA, 18 bp, Artificial
  Sythetic Primer
taccgcggctgctggcac

SEQ ID NO: 19 - DNA, 20 bp, Artificial
  Synthetic Primer
gtttgatcatggctcaggac

SEQ ID NO: 20 - DNA, 20 bp, Artificial
  Synthetic Primer
gtttgatcctggctcaggac

SEQ ID NO: 21 - DNA, 20 bp, Artificial
  Synthetic Primer
gtttgatcatggctcaggac

SEQ ID NO: 22 - DNA, 20 bp, Artificial
  Synthetic Primer
gtttgatcctggctcagaac

SEQ ID NO: 23 - DNA, 20 bp, Artificial
  Synthetic Primer
gtttgatcatggctcagaac

SEQ ID NO: 24 - DNA, 20 bp, Artificial
  Synthetic Primer
gtttgatcctggctcaggac

SEQ ID NO: 25 - DNA, 18 bp, Artificial
  Synthetic Primer
ttgatcatggctcaggac

SEQ ID NO: 26 - DNA, 18 bp, Artificial
  Synthetic Primer
ttgatcctggctcagaac

SEQ ID NO: 27 - DNA, 18 bp, Artificial
  Synthetic Primer
ttgatcatggctcagaac

SEQ ID NO: 28 - DNA, 18 bp, Artificial
  Synthetic Primer
ttgatcctggctcaggac

SEQ ID NO: 29 - DNA, 22 bp, Artificial
  Synthetic Primer
gagtttgatcatggctcagagt

SEQ ID NO: 30 - DNA, 22 bp, Artificial
  Synthetic Primer
gagtttgatcctggctcagatt

SEQ ID NO: 31 - DNA, 22 bp, Artificial
  Synthetic Primer
gagtttgatcatggctcagatt

SEQ ID NO: 32 - DNA, 22 bp, Artificial
  Synthetic Primer
gagtttgatcctggctcagagt

SEQ ID NO: 33 - DNA, 21 bp, Artificial
  Synthetic Primer
gagtttgatcatggctcagag

SEQ ID NO: 34 - DNA, 21 bp, Artificial
  Synthetic Primer
gagtttgatcctggctcagat

SEQ ID NO: 35 - DNA, 21 bp, Artificial
  Synthetic Primer
gagtttgatcatggctcagat

SEQ ID NO: 36 - DNA, 21 bp, Artificial
  Synthetic Primer
gagtttgatcctggctcagag

SEQ ID NO: 37 - DNA, 22 bp, Artificial
  Synthetic Primer
gagtttgatcatggctcagagt

SEQ ID NO: 38 - DNA, 22 bp, Artificial
  Synthetic Primer
gagtttgatcctggctcagatt

SEQ ID NO: 39 - DNA, 22 bp, Artificial
  Synthetic Primer
gagtttgatcatggctcagatt

SEQ ID NO: 40 - DNA, 22 bp, Artificial
  Synthetic Primer
gagtttgatcctggctcagagt

SEQ ID NO: 41 - DNA, 21 bp, Artificial
  Synthetic Primer
gtttgatcatggctcagagtg

SEQ ID NO: 42 - DNA, 21 bp, Artificial
  Synthetic Primer
gtttgatcctggctcagattg

SEQ ID NO: 43 - DNA, 21 bp, Artificial
  Synthetic Primer
gtttgatcatggctcagattg

SEQ ID NO: 44 - DNA, 21 bp, Artificial
  Synthetic Primer
gtttgatcctggctcagagtg

SEQ ID NO: 45 - DNA, 20 bp, Artificial
  Synthetic Primer
gtttgatcatggctcagagt

SEQ ID NO: 46 - DNA, 20 bp, Artificial
  Synthetic Primer
gtttgatcctggctcagatt

SEQ ID NO: 47 - DNA, 20 bp, Artificial
  Synthetic Primer
gtttgatcatggctcagatt

SEQ ID NO: 48 - DNA, 20 bp, Artificial
  Synthetic Primer
gtttgatcctggctcagagt

SEQ ID NO: 49 - DNA, 20 bp, Artificial
  Synthetic Primer
gtttgatcatggctcagagt

SEQ ID NO: 50 - DNA, 20 bp, Artificial
  Synthetic Primer
gtttgatcctggctcagatt

SEQ ID NO: 51 - DNA, 20 bp, Artificial
  Synthetic Primer
gtttgatcatggctcagatt

SEQ ID NO: 52 - DNA, 20 bp, Artificial
  Synthetic Primer
gtttgatcctggctcagagt

SEQ ID NO: 53 - DNA, 17 bp, Artificial
  Synthetic Primer
ttgatcatggctcagag

SEQ ID NO: 54 - DNA, 17 bp, Artificial
  Synthetic Primer
ttgatcctggctcagat

SEQ ID NO: 55 - DNA, 17 bp, Artificial
  Synthetic Primer
ttgatcatggctcagat

SEQ ID NO: 56 - DNA, 17 bp, Artificial
  Synthetic Primer
ttgatcctggctcagag

SEQ ID NO: 57 - DNA, 18 bp, Artificial
  Synthetic Primer
ttgatcatggctcagagt

SEQ ID NO: 58 - DNA, 18 bp, Artificial
  Synthetic Primer
ttgatcctggctcagatt

SEQ ID NO: 59 - DNA, 18 bp, Artificial
  Synthetic Primer
ttgatcatggctcagatt

SEQ ID NO: 60 - DNA, 18 bp, Artificial
  Synthetic Primer
ttgatcctggctcagagt

SEQ ID NO: 61 - DNA, 21 bp, Artificial
  Synthetic Primer
agtttgatcatggctcaggat

SEQ ID NO: 62 - DNA, 21 bp, Artificial
  Synthetic Primer
agtttgatcctggctcaggat

SEQ ID NO: 63 - DNA, 20 bp, Artificial
  Synthetic Primer
gtttgatcatggctcaggat

SEQ ID NO: 64 - DNA, 20 bp, Artificial
  Synthetic Primer
gtttgatcctggctcaggat

SEQ ID NO: 65 - DNA, 20 bp, Artificial
  Synthetic Primer
gtttgatcatggctcaggat

SEQ ID NO: 66 - DNA, 20 bp, Artificial
  Synthetic Primer
gtttgatcctggctcaggat

SEQ ID NO: 67 - DNA, 22 bp, Artificial Sequence
  PCR Primer
ttggagagtttgatcmtggctc

SEQ ID NO: 68 - DNA, 20 bp, Artificial Sequence
  PCR Primer
agagtttgatcmtggctcag

(19) United States
(12) Patent Application Publication
(10) Pub. No.: US 2012/0309012 A1
(43) Pub. Date: Dec. 6, 2012

(54) GROUP SPECIFIC PRIMERS

(75) Inventor: Øyvind Kommedal, Paradis (NO)

(73) Assignee: ISENTIO AS, Bergen (NO)

(21) Appl. No.: 13/509,327

(22) PCT Filed: Nov. 12, 2010
(86) PCT No.: PCT/EP2010/067417

§ 371 Date: Aug. 14, 2012

(30) Foreign Application Priority Data
Nov. 13, 2009 (GB) 0919942.3

(51) Int. Cl. C07H 21/04; C12Q 1/68

(52) U.S. Cl. 435/6.12; 536/24.33

(57) ABSTRACT

The present invention relates to group specific primers for direct 16S rDNA sequencing of polybacterial samples.

### FIELD OF THE INVENTION

The present invention relates to group specific primers for direct 16S rDNA sequencing of polybacterial samples.

### BACKGROUND

Detection and identification of bacteria directly from clinical samples by broad-range PCR targeting the 16S rRNA gene and DNA sequencing (direct 16S rDNA sequencing) is a well established method in many laboratories. It gives the possibility to identify bacteria that died during transportation or as a consequence of antibiotic treatment, and to uncover bacteria with special growth requirements. The latest advances in PCR and sequencing technology also offer a more rapid identification compared to standard phenotypical methods that depend on bacterial growth. For a review of the use of 16S rDNA for bacterial identification, see Baker et al., 2003.

Because of difficulties in the interpretation of DNA chromatograms resulting from direct sequencing of poly-bacterial samples, the use of this diagnostic tool has been limited to infections that are predominantly mono-bacterial.

Improved techniques for interpretation of complex chromatograms can assist in the direct sequencing of 16S rDNA (Kommedal et al., 2009), but there remains a need for reliable amplification of samples containing multiple species of bacteria.

### SUMMARY OF INVENTION

In a first aspect of the invention there is provided an oligonucleotide primer set for direct 16S rDNA sequencing of a polybacterial sample, comprising at least two oligonucleotide primers of formula:

5′ xz 3′

wherein x is sequence of nucleotides that hybridises to the complement of the region of the 16S rRNA gene of Escherichia coli from position 9 to position 27 and z is at least one nucleotide that provides specificity to a group of bacteria.

Preferably, z is a dinucleotide, trinucleotide or a tetranucleotide.

In a preferred embodiment, the oligonucleotide primer set for direct 16S rDNA sequencing of a polybacterial sample, consists of:
(a) an oligonucleotide primer comprising the sequence x₁gAc or x₁rAc;
(b) an oligonucleotide primer comprising the sequence x₂Akt, x₂gAk, x₂aKt or x₂aKtg; and
(c) an oligonucleotide primer comprising the sequence x₃gAt;

wherein x₁, x₂ and x₃ are each nucleotide sequences which hybridize to the complement of the region of the 16S rRNA gene of Escherichia coli from position 9 to position 27, and wherein a capital letter indicates a locked nucleic acid (LNA).

In preferred embodiments, the oligonucleotides x₁, x₂ and x₃ hybridise to the complement of the region of the 16S rRNA gene of E. coli from position 9 to position 27 under stringent conditions.

In other preferred embodiments, oligonucleotides x₁, x₂ and x₃ have at least 70%, at least 80%, at least 90% or at least 95% sequence identity to the region of the 16S rRNA gene of Escherichia coli from position 9 to position 27 (SEQ ID No. 2).

In yet other preferred embodiments, the oligonucleotides x₁, x₂ and x₃ have from 14 to 19 consecutive bases from the sequence defined in SEQ ID No. 2 with 0, 1, 2, 3, 4 or 5 mismatches.

In another preferred embodiment, the oligonucleotide primer set consists of an oligonucleotide primer consisting of the nucleotide sequence of any one of SEQ ID Nos. 3 to 5; an oligonucleotide primer consisting of the nucleotide sequence of any one of SEQ ID Nos. 6 to 13; and an oligonucleotide primer consisting of the nucleotide sequence of any one of SEQ ID Nos. 14 to 16.

A particularly preferred primer set consists of an oligonucleotide primer consisting of the nucleotide sequence of SEQ ID No. 4; an oligonucleotide primer consisting of the nucleotide sequence of SEQ ID No. 9; and an oligonucleotide primer consisting of the nucleotide sequence of SEQ ID No. 14. Another preferred primer set consists of an oligonucleotide primer consisting of the nucleotide sequence of SEQ ID No. 3; an oligonucleotide primer consisting of the nucleotide sequence of SEQ ID No. 8; and an oligonucleotide primer consisting of the nucleotide sequence of SEQ ID No. 14.

In a second aspect of the present invention, there is provided a kit for direct 16S rDNA sequencing of a polybacterial sample, comprising at least two oligonucleotide primers of formula:

5′ xz 3′

where x is a sequence of nucleotides that hybridises to the complement of the region of the 16S rRNA gene of Escherichia coli from position 9 to position 27 and z is at least one nucleotide that provides specificity to a group of bacteria.

Preferably, z is a dinucleotide, trinucleotide or a tetranucleotide.

In preferred embodiments, the kit for direct 16S rDNA sequencing of a polybacterial sample, comprises:
(a) an oligonucleotide primer comprising the sequence x₁gAc or x₁rAc;
(b) an oligonucleotide primer comprising the sequence x₂Akt, x₂gAk, x₂aKt or x₂aKtg; and
(c) an oligonucleotide primer comprising the sequence x₃gAt;

wherein x₁, x₂ and x₃ are each nucleotide sequences which hybridize to the complement of the region of the 16S rRNA gene of Escherichia coli from position 9 to position 27, and wherein a capital letter indicates a locked nucleic acid (LNA).

In preferred embodiments, the oligonucleotides x₁, x₂ and x₃ hybridise to the complement of the region of the 16S rRNA gene of E. coli from position 9 to position 27 under stringent conditions.

In other preferred embodiments, oligonucleotides x₁, x₂ and x₃ have at least 70%, at least 80%, at least 90% or at least 95% sequence identity to the region of the 16S rRNA gene of Escherichia coli from position 9 to position 27 (SEQ ID No. 2).

In yet other preferred embodiments, the oligonucleotides x₁, x₂ and x₃ have from 14 to 19 consecutive bases from the sequence defined in SEQ ID No. 2 with 0, 1, 2, 3, 4 or 5 mismatches.

In another preferred embodiment, the kit comprises an oligonucleotide primer consisting of the nucleotide sequence of any one of SEQ ID Nos. 3 to 5; an oligonucleotide primer consisting of the nucleotide sequence of any one of SEQ ID Nos. 6 to 13; and an oligonucleotide primer consisting of the nucleotide sequence of any one of SEQ ID Nos. 14 to 16.

Preferably, the kit comprises an oligonucleotide primer consisting of the nucleotide sequence of SEQ ID No. 4; an oligonucleotide primer consisting of the nucleotide sequence of SEQ ID No. 9; and an oligonucleotide primer consisting of the nucleotide sequence of SEQ ID No. 14. Another preferred kit comprises an oligonucleotide primer consisting of the nucleotide sequence of SEQ ID No. 3; an oligonucleotide primer consisting of the nucleotide sequence of SEQ ID No. 8; and an oligonucleotide primer consisting of the nucleotide sequence of SEQ ID No. 14.

In another preferred embodiment, the kit of the present invention further comprises a reverse oligonucleotide primer. Exemplary reverse primers include but are not limited to those provided in SEQ ID No. 17 and SEQ ID No. 18. Either one of these reverse primers can be used in conjunction with forward primers in the kit as defined above to form functional primer-pairs.

In a third aspect of the present invention, there is provided method of direct sequencing of 16S rDNA in a polybacterial sample, the method comprising the steps of:
(a) splitting the sample into at least two aliquots;
(b) conducting a first PCR amplification with a first aliquot using a first forward primer of formula 5′ xz 3′ and a suitable reverse primer;
(c) conducting a second PCR amplification with a second aliquot using a second forward primer of formula 5′ xz 3′, wherein the second forward primer is of different sequence to the first forward primer and a suitable reverse primer; and
(d) sequencing the PCR products obtained in steps (b) to (c) using suitable sequencing primers;
   wherein x is an oligonucleotide which hybridizes to the complement of the region of the 16S rRNA gene of Escherichia coli from position 9 to position 27, and wherein z is at least one nucleotide that provides specificity to a group of bacteria, wherein the first and second forward primers are specific for different groups of bacteria, and wherein steps (b) to (c) may be carried out concurrently or consecutively in any order.

In preferred embodiments the method comprising the steps of:
(a) splitting the sample into at least three aliquots;
(b) conducting a PCR amplification with a first aliquot using forward primers comprising the sequence x₁gAc or x₁rAc and a suitable reverse primer;
(c) conducting a PCR amplification with a second aliquot using forward primers comprising the sequence x₂Akt, x₂gAk, x₂aKt or x₂aKtg and a suitable reverse primer;
(d) conducting a PCR amplification with a third aliquot using forward primers comprising the sequence x₃gAt and a suitable reverse primer; and
(e) sequencing the PCR products obtained in steps (b) to (d) using suitable sequencing primers;

wherein x₁, x₂ and x₃ are oligonucleotides which hybridize to the complement of the region of the 16S rRNA gene of Escherichia coli from position 9 to position 27, and wherein a capital letter indicates a locked nucleic acid (LNA), and wherein steps (b) to (d) may be carried out concurrently or consecutively in any order.

Preferably, z is a dinucleotide, trinucleotide or a tetranucleotide. PCR amplifications (b), (c) and (d) are typically carried out in separate reaction vessels, e.g. separate PCR reaction tubes or separate wells of a microtiter plate.

In preferred embodiments, the oligonucleotides x₁, x₂ and x₃ hybridise to the complement of the region of the 16S rRNA gene of E. coli from position 9 to position 27 under stringent conditions.

In another preferred embodiment, the primers for steps (b) to (d) are selected from: (a) a primer consisting of the nucleotide sequence of any one of SEQ ID Nos. 3 to 5; (b) a primer consisting of the nucleotide sequence of any one of SEQ ID Nos. 6 to 13; and (c) a primer consisting of the nucleotide sequence of any one of SEQ ID Nos. 14 to 16.

Preferably, the primers for steps (b) to (d) are: (a) a primer consisting of the nucleotide sequence of SEQ ID No. 4; (b) a primer consisting of the nucleotide sequence of SEQ ID No. 9; and (c) a primer consisting of the nucleotide sequence of SEQ ID No. 14. In another embodiment the primers for steps (b) to (d) are: (a) a primer consisting of the nucleotide sequence of SEQ ID No. 3; (b) a primer consisting of the nucleotide sequence of SEQ ID No. 8; and (c) a primer consisting of the nucleotide sequence of SEQ ID No. 14.

In other preferred embodiments, oligonucleotides x₁, x₂ and x₃ have at least 70%, at least 80%, at least 90% or at least 95% sequence identity to the region of the 16S rRNA gene of Escherichia coli from position 9 to position 27 (SEQ ID No. 2).

In yet other preferred embodiments, the oligonucleotides x₁, x₂ and x₃ have from 14 to 19 consecutive bases from the sequence defined in SEQ ID No. 2 with 0, 1, 2, 3, 4 or 5 mismatches.

### DETAILED DESCRIPTION

Amplification of target DNA followed by DNA sequencing directly from a sample is now a commonly used laboratory technique. After any appropriate sample preparation steps, the region of DNA of interest is amplified by PCR using appropriate forward and reverse primers. The sequence of the amplified region is then determined by standard sequencing techniques. The sequencing reaction requires only a single forward or reverse primer, which may be identical to the forward primer used for PCR amplification. Alternatively, a dedicated sequencing primer (or primers) may be used.

Direct sequencing of the 16S rRNA gene allows identification of bacterial species due to the variability in sequence of the 16S rRNA gene. In order to amplify the region of interest, primers can be designed to hybridise to conserved regions of the 16S rRNA gene. Such primers may be described as ‘universal’, since they are capable of hybridising to the 16S rRNA gene in a large number of different bacterial species. For a review of the design of such primers, see Baker et al., 2003.

Direct sequencing of polybacterial samples using universal primers results in complex chromatograms that are difficult to interpret. Furthermore, samples containing large amounts of a first bacteria and a smaller population of a second bacteria may result in partly false negative results where the universal primer targets the 16S rRNA gene of both bacteria, since the lower concentration bacteria will be outcompeted in the PCR reaction.

In order to overcome these problems the present inventors realised that, rather than attempt to amplify all relevant bacteria which may be present in a test sample with a single pair of universal primers, it would instead be advantageous to split the test sample into two or more aliquots and then carry out a separate amplification reaction on each aliquot of the sample, each separate amplification reaction using a different pair of group- specific primers (or at least a different group-specific forward primer). “Group specific primers” are primers that target particular groups of bacteria, i.e. primers capable of hybridising to the 16S rRNA gene from a group of bacterial species. In this context a “group” of bacteria represents a subset of the complete set of bacterial species which may be (or are expected to be) present in a given test sample. In this manner, the competition for primers will be reduced and the complexity of the chromatograms produced in sequencing reactions carried out on the products of each separate amplification reaction is much reduced. In order to get the statistically largest probability that the various species in a sample are evenly distributed between the different group specific primers, the populations targeted by each of the primers should be equal or similar in size.

### Definitions

“Primer” shall be taken to mean any single-stranded oligonucleotide capable of priming a polymerase.

Where a primer is defined as having an ambiguous position, identified for example by the letters r (g or a), y (t/u or c), m (a or c), k (g or t/u), s (g or c), w (a or t/u), b (g or c or t/u), d (a or g or t/u), h (a or c or t/u), v (a or g or c), or n (a or g or c or t/u), it will be understood that the primer is made up of equal quantities of oligonucleotide molecules having each possible base. Alternatively, primers defined as having an ambiguous position may incorporate nucleotides having bases that bind to the bases defined by the ambiguous position equally well. For example, inosine will base-pair to a, t or e.

A “universal primer” is one designed to hybridise to a large number of varying sequences. In the context of the present application, such universal primers are intended to hybridise to the 16S rRNA gene of any bacterial species.

A “group specific primer” is a primer designed to hybridise to a group of related sequences. In the context of the present application, such group specific primers are intended to hybridise to the 16S rRNA gene from a particular group or subset of bacterial species.

“Primer set” means a set of at least two primers. The primers within the set may be of the same orientation, i.e. all may be “forward” primers.

“Primer pair” means a pair of forward and reverse primers which form a functional pairing, i.e. they can be used for PCR amplification of a target nucleic acid.

The meaning of the phrase “stringent conditions” will be clear to those of skill in the art. Generally, it is intended to encompass the conditions found during the annealing step of a typical PCR. The conditions encountered during the annealing steps of a PCR will be generally known to one skilled in the art, although the precise annealing conditions will vary from reaction to reaction (see Sambrook et al., 2001, Molecular Cloning, A Laboratory Manual, 3rd Ed, Cold Spring Harbor Laboratory Press, NY; Current Protocols, eds Ausubel et al.). Typically such conditions may comprise, but are not limited to, (following a denaturing step at a temperature of about 94° C. for about one minute) exposure to a temperature in the range of from 40° C. to 72° C. (preferably 50-68° C.) for a period of about 1 minute in standard PCR reaction buffer.

The term “locked nucleic acid” or “LNA” will be understood by those of skill in the art to refer to a modified ribonucleotide in which the 2′ and 4′ carbons of the ribose moiety are connected by an additional bridge. Throughout the present application, the use of a LNA is indicated by the use of a capital letter. “Direct 16S rDNA sequencing” is the process of determining a 16S rRNA gene sequence from a sample. Briefly, DNA is prepared from the sample in the standard fashion. A portion of the 16S rRNA gene is then amplified by PCR using a forward and reverse primer. The amplified PCR products may then be sequenced by standard methods. The forward and/or reverse primers in the PCR amplification stage may be used as sequencing primers. Alternatively, a dedicated sequencing primer or primers may be used.

“Polybacterial sample” means any sample that is expected to contain multiple bacterial species or strains. Examples include, but are not limited to, environmental samples such as river, pond, lake, sea or waste water and clinical samples such as abscesses in internal organs (brain, lung, spleen, liver, pancreas, kidney, ovaries, aorta), deep soft-tissue or muscular abscesses, retroperitoneal abscesses, aspirate/biopsies from spondylodiscitis and other bone-related infections, pus, pleural fluids, blood, bile, urine or saliva.

“Monobacterial sample” means any sample expected to contain only a single bacterial species (for example synovial fluids, cerebrospinal fluids or pericardial fluids).

The present invention provides primer sets, in particular sets of forward primers, for direct 16S rDNA sequencing of a poly-bacterial sample. The primer set comprises at least two oligonucleotide primers. Preferably, the primer set comprises or consists of from 4 to 10 primers. More preferably, the primer set comprises or consists of three oligonucleotide primers. The primers in the primer set are designed such that amplification of the 16S rRNA gene of all relevant bacteria is equally divided between the primers in the set. This ensures that all pathogens are amplified but reduces competition for primers in each reaction.

Specificity for a group of bacteria is achieved via the 3′ end of the primer. Each primer in the set is specific for a (different) group of bacterial species. Preferably the groups do not overlap to any significant extent. The sequence of the 5′ portion of the primer may be varied, provided that sufficient hybridising power is maintained to ensure correct priming of the polymerase enzyme with which the primers are to be used (e.g. Taq polymerase for PCR amplification).

The primers of the invention have the following general formula (I):

5′ xz 3′ (I)

Where x is an oligonucleotide that hybridises to the complement of the region of the 16S rRNA gene of Escherichia coil from position 9 to position 27 (the E. coli 16S rRNA gene sequence is provided in SEQ ID No. 1; the region from position 9 to position 27 is given in SEQ ID No. 2) and z is at least one nucleotide that provides specificity to a group of bacteria. Preferably z is a dinucleotide, more preferably a trinucleotide. In certain embodiments, z comprises four or more nucleotides. In preferred embodiments z may be a sequence of nucleotides that hybridises to the complement of the region of the 16S rRNA gene of Escherichia coli from position 28 to position 31.

In preferred embodiments, the oligonucleotide x hybridises to the complement of the region of the 16S rRNA gene of E. coli from position 9 to position 27 under stringent conditions, as defined above.

In other preferred embodiments, oligonucleotide x has at least 70%, at least 80%, at least 90% or at least 95% sequence identity to the region of the 16S rRNA gene of Escherichia coli from position 9 to position 27 (SEQ ID No. 2).

In yet other preferred embodiments, the oligonucleotide x may include from 14 to 19 consecutive bases from the 3′ end of the sequence defined in SEQ ID No. 2. Within this region one or more mismatches (e.g. 1, 2 or 3 mismatches) with the sequence shown in SEQ ID No. 2 may be tolerated without altering the performance of the primer (e.g. in PCR) to a material extent. Therefore, primers incorporating mismatches with SEQ ID No.2 also form part of the subject-matter of the invention.

In preferred embodiments of the invention, the primers may contain one or more locked nucleic acids (LNAs) in region z. LNAs increase the thermal stability of primers. The presence of an LNA increases the effect of single nucleotide mismatches at either the LNA or LNA+1 positions. It will be clear to those of skill in the art that the term ‘LNA+1’ indicates the next nucleotide in the 5′ to 3′ direction. In addition, a LNA three bases upstream of the 3′ end of a primer (i.e., three bases from the 3′ terminus in the 3′ to 5′ direction) protects the nucleotide at the 3′ terminal position from the 3′-5′ exonuclease activity of some polymerases.

The use of LNAs in the present primers allows the nucleotide(s) defined as z in formula I to provide the necessary specificity (for a group of bacterial species). Region x may also contain LNAs if this leads to primers with more appropriate melting temperatures (Tₘ). Throughout the present application, the use of a LNA is indicated by capitalisation.

Exemplary primers are given in table 1.

TABLE 1
Exemplary primers.

| E. Coli 16S sequence: | | SEQ ID No. |
|---|---|---|
| 5′ gagtttgatcctggctcag | attg 3′ | 2 |
| 5′ x | z 3′ | |
| Group A | | |
| 5′ --gtttgatcmtggctcag | gAc 3′ | 3 |
| 5′ --gttTgatcmtggctcag | rAc 3′ | 4 |
| 5′ ----ttgatcmtggctcag | rAc 3′ | 5 |
| Group B | | |
| 5′ gagtttgatcmtggctcag | Akt 3′ | 6 |
| 5′ gagtttgatcmtggctcag | Ak 3′ | 7 |
| 5′ gagtttgatcmtggctcag | aKt 3′ | 8 |
| 5′ --gttTgatcmtggctcag | aKtg 3′ | 9 |
| 5′ --gttTgatcmtggctcag | aKt 3′ | 10 |
| 5′ --gtttgatcmtggctcag | aKt 3′ | 11 |
| 5′ ----tTgATcmtggctcag | Ak 3′ | 12 |
| 5′ ----tTgATcmtggctcag | aKt 3′ | 13 |
| Group C | | |
| 5′ -agtttgatcmtggctcag | gAt 3′ | 14 |
| 5′ --gTtTgatcmtggctcag | gAt 3′ | 15 |
| 5′ --gtttgatcmtggctcag | gAt 3′ | 16 |

Key:
m = a or c, r = g or a, k = g or t, - = gap, a capital letter indicates a locked nucleic acid (LNA)

Each of the primers in groups a, b and c as listed in table 1 are intended to amplify the 16S rRNA gene from a different group of bacteria. In this context, a group of bacteria simply means a subset of the entire bacterial population expected or anticipated to be present in the sample. In order to prevent competition for primers, the entire bacterial population should distribute evenly between groups a, b and c. Thus, groups a, b and c will be of approximately equal size. In addition, the content of groups a, b and c should not overlap to any significant extent. A primer set can be created by selecting one primer from each of groups a, b and c.

The 16S rRNA gene from a first bacteria in the sample will be amplified by a primer from group a but not from groups b or c. The 16S rRNA gene from a second bacteria in the sample may be amplified by a primer from group b but not the primers from groups a and c. Since the reactions using primers a, b and c are carried out separately, the amplification of the 16S rRNA gene from the first bacteria does not compete with the amplification of the 16S rRNA gene from the second bacteria. Thus, the presence of both the first and second bacteria can be reliably detected, even where one is present at a significantly higher concentration than the other.

Where an ambiguous base is indicated in a primer sequence, for example by the letters m, r or k, it will be clear to those of skill in the art that efficient hybridisation will be achieved by either (a) including equal amounts of primers containing each possible base; or (b) including at that position a nucleotide incorporating a base that base-pairs to each possible base equally well. For example, nucleotides incorporating inosine base-pair to a, t or c.

For the purposes of clarity, the primers in Table 1 are also listed in Table 2 together with primers with the ambiguous bases replaced by their possible identities. As an example, the primer of SEQ ID No. 3 may be composed of equal amounts of oligonucleotides with the sequences of SEQ ID Nos. 19 and 20. Alternatively, inosine may be incorporated at position 9 in the primer, which will base pair with a, t or c in the target sequence.

TABLE 2
Non-ambiguous primers.

| SEQ ID | Sequence | SEQ ID | Sequence |
|---|---|---|---|
| 2 | gagtttgatcctggctcagattg | 2 | gagtttgatcctggctcagattg |
| 3 | --gtttgatcmtggctcaggAc- | 10 | --gttTgatcmtggetcagaKt- |
| 19 | --gtttgatcatggctcaggAc- | 45 | --gttTgatcatggctcagaGt- |
| 20 | --gtttgatcctggctcaggAc- | 46 | --gttTgatcctggctcagaTt- |
| 4 | --gttTgatcmtggctcagrAc- | 47 | --gttTgatcatggctcagaTt- |
| 21 | --gttTgatcatggctcaggAc- | 48 | --gttTgatcctggctcagaGt- |
| 22 | --gttTgatcctggctcagaAc- | 11 | --gtttgatcmtggctcagaKt- |
| 23 | --gttTgatcatggctcagaAc- | 49 | --gtttgatcatggctcagaGt- |
| 24 | --gttTgatcctggctcaggAc- | 50 | --gtttgatcctggctcagaTt- |
| 5 | ----ttgatcmtggctcagrAc- | 51 | --gtttgatcatggctcagaTt- |
| 25 | ----ttgatcatggctcaggAc- | 52 | --gtttgatcctggctcagaGt- |
| 26 | ----ttgatcctggctcagaAc- | 12 | ----tTgATcmtggctcagAk-- |
| 27 | ----ttgatcatggctcagaAc- | 53 | ----tTgATcatggctcagAg-- |
| 29 | ----ttgatcctggctcaggAc- | 54 | ----tTgATcctggctcagAt-- |
| 6 | gagtttgatcmtggctcagAkt- | 55 | ----tTgATcatggctcagAt-- |
| 25 | gagtttgatcatggctcagAgt- | 56 | ----tTgATcctggctcagAg-- |
| 30 | gagtttgatcctggctcagAtt- | 13 | ----tTgATcmtggctcagaKt- |
| 31 | gagtttgatcatggctcagAtt- | 57 | ----tTgATcatggctcagaGt- |
| 32 | gagtttgatcctggctcagAgt- | 58 | ----tTgATcctggctcagaTt- |
| 7 | gagtttgatcmtggctcagAk-- | 59 | ----tTgATcatggctcagaTt- |
| 33 | gagtttgatcatggctcagAg-- | 60 | ----tTgATcctggctcagaGt- |
| 34 | gagtttgatcctggctcagAt-- | 14 | -agtttgatcmtggctcaggAt- |
| 35 | gagtttgatcatggctcagAt-- | 61 | -agtttgatcatggctcaggAt- |
| 36 | gagtttgatcctggctcagAg-- | 62 | -agtttgatcctggctcaggAt- |
| 8 | gagtttgatcmtggctcagaKt- | 15 | --gTtTgatcmtggctcaggAt- |
| 37 | gagtttgatcatggctcagaGt- | 63 | --gTtTgatcatggctcaggAt- |
| 38 | gagtttgatcctggctcagaTt- | 64 | --gTtTgatcctggctcaggAt- |
| 39 | gagtttgatcatggctcagaTt- | 16 | --gtttgatcmtggctcaggAt- |
| 40 | gagtttgatcctggctcagaGt- | 65 | --gtttgatcatggctcaggAt- |
| 9 | --gttTgatcmtggctcagaKtg | 66 | --gtttgatcctggctcaggAt- |
| 41 | --gttTgatcatggctcagaGtg | | |
| 42 | --gttTgatcctggctcagaTtg | | |
| 43 | --gttTgatcatggctcagaTtg | | |
| 44 | --gttTgatcctggctcagaGtg | | |

Key:
m = a or c, r = g or a, k = g ort, - = gap, a capital letter indicates a locked nucleic acid (LNA)

One primer from each of groups a, b and c in Table 1 is selected to form a set of three primers. Preferred primer sets include primer sets comprising or consisting of primers with sequences as shown in SEQ ID Nos. 3, 6 & 14; SEQ ID Nos. 3, 7 & 14; SEQ ID Nos. 3, 8 & 14; SEQ ID Nos. 3, 9 & 14; SEQ ID Nos. 3, 10 & 14; SEQ ID Nos. 3, 11 & 14; SEQ ID Nos. 3, 12 & 14; SEQ ID Nos. 3, 13 & 14; SEQ ID Nos. 3, 6 & 15; SEQ ID Nos. 3, 7 & 15; SEQ ID Nos. 3, 8 & 15; SEQ ID Nos. 3, 9 & 15; SEQ ID Nos. 3, 10 & 15; SEQ ID Nos. 3, 11 & 15; SEQ ID Nos. 3, 12 & 15; SEQ ID Nos. 3, 13 & 15; SEQ ID Nos. 3, 6 & 16; SEQ ID Nos. 3, 7 & 16; SEQ ID Nos. 3, 8 & 16; SEQ ID Nos. 3, 9 & 16; SEQ ID Nos. 3, 10 & 16; SEQ ID Nos. 3, 11 & 16; SEQ ID Nos. 3, 12 & 16; SEQ ID Nos. 3, 13 & 16; SEQ ID Nos. 4, 6 & 14; SEQ ID Nos. 4, 7 & 14; SEQ ID Nos. 4, 8 & 14; SEQ ID Nos. 4, 9 & 14; SEQ ID Nos. 4, 10 & 14; SEQ ID Nos. 4, 11 & 14; SEQ ID Nos. 4, 12 & 14; SEQ ID Nos. 4, 13 & 14; SEQ ID Nos. 4, 6 & 15; SEQ ID Nos. 4, 7 & 15; SEQ ID Nos. 4, 8 & 15; SEQ ID Nos. 4, 9 & 15; SEQ ID Nos. 4, 10 & 15; SEQ ID Nos. 4, 11 & 15; SEQ ID Nos. 4, 12 & 15; SEQ ID Nos. 4, 13 & 15; SEQ ID Nos. 4, 6 & 16; SEQ ID Nos. 4, 7 & 16; SEQ ID Nos. 4, 8 & 16; SEQ ID Nos. 4, 9 & 16; SEQ ID Nos. 4, 10 & 16; SEQ ID Nos. 4, 11 & 16; SEQ ID Nos. 4, 12 & 16; SEQ ID Nos. 4, 13 & 16; SEQ ID Nos. 5, 6 & 14; SEQ ID Nos. 5, 7 & 14; SEQ ID Nos. 5, 8 & 14; SEQ ID Nos. 5, 9 & 14; SEQ ID Nos. 5, 10 & 14; SEQ ID Nos. 5, 11 & 14; SEQ ID Nos. 5, 12 & 14; SEQ ID Nos. 5, 13 & 14; SEQ ID Nos. 5, 6 & 15; SEQ ID Nos. 5, 7 & 15; SEQ ID Nos. 5, 8 & 15; SEQ ID Nos. 5, 9 & 15; SEQ ID Nos. 5, 10 & 15; SEQ ID Nos. 5, 11 & 15; SEQ ID Nos. 5, 12 & 15; SEQ ID Nos. 5, 13 & 15; SEQ ID Nos. 5, 6 & 16; SEQ ID Nos. 5, 7 & 16; SEQ ID Nos. 5, 8 & 16; SEQ ID Nos. 5, 9 & 16; SEQ ID Nos. 5, 10 & 16; SEQ ID Nos. 5, 11 & 16; SEQ ID Nos. 5, 12 & 16; or SEQ ID Nos. 5, 13 & 16.

Particularly preferred primer sets include, but are not limited to, primer sets comprising or consisting of primers with sequences as shown in SEQ ID Nos. 3, 8 and 14 or 4, 9 and 14.

Separate PCR amplifications may be carried out using these primers as forward primers, together with a suitable reverse primer. Sequencing may then be carried out also using the same forward and/or reverse primers. Although the present application focuses on the use of primer sets made up of three primers, it is of course possible to design a primer set that comprises 4, 5, 6 or more primers, each of which is directed to a separate subset of the potential bacterial population.

The primers described herein are suitable for use in direct 16S rDNA sequencing of clinical samples. However, they also find utility in the PCR amplification of mono and/or polybacterial samples and in the sequencing of DNA.

It will be clear to those of skill in the art that suitable primer sets, kits and methods may use any number of primers, so long as the population of bacteria to be amplified are evenly distributed between the primers. Accordingly, the present invention is not limited to the use of three primers as presently exemplified.

### EXAMPLE 1

In order to test the performance of the primers from each of groups A, B and C, laboratory tests were carried out.

The ability of the designed primers to act as forward primers during PCR of a polybacterial sample was explored.

### Preparation of Bacterial DNA

A mixture of DNA from Enterococcus faecalis (EF), Escherichia coli (EC) and Staphylococcus aureus (SA) was prepared as the test polybacterial sample.

From fresh culture solutions of 0.5 McFarland (≈300×10⁶ colony forming units per mL) of EC, EF and SA were prepared. 800 μL of each solution was added to separate lysis tubes and lyzed mechanically using a Roche MagNaLyzer instrument. The tubes were then centrifuged for 3 minutes at 13,000 rpm. The supernatant was removed by pipette and used directly as template in the subsequent PCR.

### Real-Time PCR

Real-time PCR analysis was carried out using the following protocol.

### Primers:

| | |
|---|---|
| Forward: | Primer A: SEQ ID No. 3: matches the EF 16S rRNA gene |
| | (Tₘ = 64° C.) |
| | Primer B: SEQ ID No. 8: matches the EC 16S rRNA gene |
| | (Tₘ = 62° C.) |
| | Primer C: SEQ ID No. 14: matches the SA 16S rRNA gene |
| | (Tₘ = 64° C.) |

Reverse (Bosshard et al): 5′-gta-tta-ccg-cgg-ctg-ctg-3′ (SEQ ID No. 17)

The resulting product has a size of approximately 510 base pairs, covering the variable areas V1, V2 and V3 of the 16S rRNA gene (Baker et al.).

### Mixture:

The following mixture was used for all PCR amplifications:

TABLE 2
PCR mixture components

| | |
|---|---|
| SYBR Premix Ex Taq (TaKaRa) | 12.5 μl |
| Forward primer (5 μM) | 2.0 μl |
| Reverse primer (5 μM) | 1.0 μl |
| H₂O (PCR grade) | 7.5 μl |
| EC Template (EC or EF or SA) | 2.0 μl |
| Total volume | 25.0 μl |

SYBR Premix Ex Taq is commercially available from TaKaRa Bio, Inc.

### Conditions:

The real-time PCR was run on a SmartCycler machine (Cepheid) for 40 cycles using the following conditions:

TABLE 3
PCR conditions

| | | |
|---|---|---|
| Initial Enzyme activation | 95° C. | 10 seconds |
| Melting | 95° C. | 10 seconds |
| Annealing | 62/64° C. | 15 seconds |
| Extension | 72° C. | 20 seconds |

### Interpretation of PCR Results:

A positive sample is detected if the fluorescence intensity reaches threshold value ≧3 cycles before the negative control.

Samples that never reach threshold are likely to contain inhibitory substances or inhibitory amounts of DNA. These samples are diluted 1:10 and re-run.

Samples reaching threshold less than 3 cycles before the negative control, or even after the negative control, are also sequenced if the melt point analysis shows a tall distinct peak significantly different from the pattern seen in the negative control. The result should be interpreted with caution and taken into consideration only if well known relevant human pathogens are detected.

### Results

When using primer A in the PCR amplification of the EF/EC/SA DNA mixture, the EF PCR product crossed the desired fluorescence threshold (30.0) 18.1 PCR cycles before EC or SA.

Similarly, PCR amplification of the EF/EC/SA mixture with primer B the EC PCR product crossed the fluorescence threshold (30.0) 16.3 PCR cycles before either EF or SA.

When primer C is used in the PCR amplification of a mixture of equal amounts of EF and SA DNA, the SA PCR product reached the fluorescence threshold (30.0) 10.7 cycles before the EF PCR product. When primer C is used in the PCR amplification of a mixture of equal amounts of EC and SA DNA, the SA PCR product reached the fluorescence threshold (30.0) 26.9 cycles before the EC PCR product.

These data demonstrate that the ‘incorrect’ primer requires between 10³ and 10⁸ times the amount of DNA to reach the fluorescence threshold (30.0) in the same number of cycles as the matching primer.

### Cycle Sequencing

The PCR products obtained may be sequenced according to the following protocol:

### Primer:

Reverse CLSI:
(SEQ ID No. 18)
5′-tac-cgc-ggc-tgc-tgg-cac-3′.

### PCR Clean Up:

The PCR products from the positive samples are purified with ExoSap-IT (Affymetrix). After ExoSap-IT treatment, the purified PCR products are diluted 1:3 (1 part product, 2 parts water). The diluted product is used as template in the following cycle sequencing mixture:

### Mixture:

TABLE 4
Sequencing mixture components

| | |
|---|---|
| BigDye version 1.1 | 1.0 μl |
| Seq. buffer | 2.0 μl |
| Primer (5 μM) | 1.0 μl |
| H₂O (PCR grade) | 5.0 μl |
| template | 1.0 μl |
| Total volume | 10.0 μl |

The ABI PRISM Big-dye sequencing kit is commercially available from Applied Biosystems.

### Cycling Conditions:

We use 60° C. as annealing temperature in the cycle sequencing reaction, and run it for 30 cycles using a 3730 DNA Analyzer (Applied Biosystems).

### EXAMPLE 2

The performance of the group-specific primers in clinical samples was tested as described below.

### Materials and Methods:

### Cloning and Plasmid Isolation

The 16S rRNA genes from relevant bacterial strains were amplified using primers 4f+1542r. The PCR products were cloned into pCR 2.2 TOPO® vector and transformed into competent E. coli cells using the TOPO TA cloning kit (Invitrogen, Carlsbad, Calif.) following the manufacturer's protocol. E. coli containing verified clones were cultured aerobically overnight. Plasmids were isolated from 2 ml E. coli culture using PureLink™ Quick Plasmid Miniprep Kit (Invitrogen). The isolated plasmids were eluted to 10¹⁰/μl and frozen at −20° C. until they were used.

### Clinical Samples

The following poly-microbial specimens were included together with their corresponding negative controls: Brain abscess (n=5), bile (n=2), empyema (n=6), liver abscess (n=3), ovarian abscess (n=1), pancreas abscess (n=2), para-aortic abscess (n=1), retro-peritoneal abscess (n=2), soft tissue abscess (n=2), spleen abscess (n=1). In addition 25 presumably negative specimens and their controls were included: Various abscess or abscess-like specimens (n=5), bone biopsy (n=1), Pericardial fluid (n=3), periprosthetic tissue (n=3), peritoneal fluid (n=2), pleural fluid (n=6), subdural hematoma (n=1) and synovial fluid (n=4). All samples were investigated using the three separate group-specific real-time PCRs with SYBR-green detection followed by DNA sequencing. In parallel all samples were analyzed using a single universal primer pair targeting the same part of the 16S rRNA gene as the group specific primers (first 500 bp).

### Pre-PCR Treatment

The samples had been extracted using the following protocol: Between 200 and 800 μl sample material was added to a bead containing tube (SeptiFast Lysis Kit, Roche, Mannheim, Germany), together with 400 μl of Bacterial Lysis Buffer (BLB, Roche). Eight-hundred μl was the maximum capacity of the bead-tube, and used for liquid samples with low viscosity. For other samples 400 μl were used if available. Two hundred μl was the lowest volume that would still provide 400 μl of supernatant for the subsequent DNA purification and was the lowest volume accepted for all specimens. A negative control containing lysis buffer and 400 μl of PCR-grade water was included in every batch of samples. The samples were run for 2×45 seconds in a FastPrep machine (Cepheid, Sunnyvale, Calif.) at speed 6.5. After a short spin, 400 μl of supernatant were transferred to a MagNa Pure Compact automated extractor (Roche) and DNA extracted and purified using the “total nucleic acid” program according to the manufacturer's instructions. The resulting 50 μl of eluate was stored at −80° C. until used.

### Primers Used for PCRs:

Primer pair universal PCR:

Forward (CLSI):
(SEQ ID No. 67)
5′-ttg-gag-agt-ttg-atc-mtg-gct-c-3′
(11)
Reverse (Bosshard):
(SEQ ID No. 17)
5′-gta-tta-ccg-cgg-ctg-ctg-3′
(5, 6)

Proprietary primer pairs group-specific PCRs (iSentio, Norway):

Primer pair A:
(SEQ ID No. 4)
5′-gttTgatcmtggctcagrAc-3′
(Gram-positive cocci in chains and
non-anaerobic Gram-positive rods)
Primer pair B:
(SEQ ID No. 9)
5′-gttTgatcmtggctcagaKtg-3′;
(Non-anaerobic Gram-negative bacteria)
Primer pair C:
(SEQ ID No. 14)
5′-agtttgatcmtggctcaggAt-3′.
(Staphylococcus spp. and anaerobic bacteria
not covered for by primer A)

### Primers Used in Cycle Sequencing Reactions:

### Universal Reaction:

Forward (Bosshard):
(SEQ ID No. 68)
5′-aga-gtt-tga-tcm-tgg-ctc-ag-3′
Reverse (Bosshard):
(SEQ ID No. 17)
5′-gta-tta-ccg-cgg-ctg-ctg-3′

Group Specific Reactions:

Common primer R:
(SEQ ID No. 18)
5′-tac-cgc-ggc-tgc-tgg-cac-3′

### PCR-Conditions

All PCR reactions were performed in 25 μl reaction tubes on a real-time SmartCycler apparatus (Cepheid). The universal PCR mixture consisted of 12.5 μl ExTaq SYBR master mix (TaKaRa, Japan), 0.4 μM F-primer and 0.4 μM R-primer, 8.5 μl PCR-grade water and 2 μl template. All group specific PCR mixtures consisted of 12.5 μl ExTaq SYBR master mix, 0.8 μM F-primer and 0.4 μM R-primer, 7.5 μl PCR-grade water and 2 μl template. The PCR thermal profile was identical for all reactions and included an initial polymerase activation step of 10 seconds at 95° C. followed by 40 cycles of 15 s at 95° C., 10 s at 64° C. and 20 s at 72° C.

### Negative Controls:

For all PCRs every sample was run in parallel with its negative control. For the universal PCR a positive sample was defined if fluorescence threshold value (Ct) was reached >=3 cycles ahead of the negative control. For the group specific PCRs the negative control that became positive first for a given sample defined the cut-off for positivity for all three group-specific PCRs for that sample (i.e. >=3 cycles before earliest negative control).

### Sequencing

The amplicons from the positive PCR reactions were spun out of the Smart Cycler reaction tubes into a 1.5 ml Eppendorf tube and cleaned up using the ExoSAP-IT enzymatic degradation kit (Affymetrix). Sequencing was performed in a core facility using the ABI PRISM 1.1 Big-dye sequencing kit and a 3730 DNA Analyzer (Applied Biosystems, Foster City, Calif.).

### Interpretation of Chromatograms

Mixed DNA-chromatograms were analyzed using the RipSeq Mixed web-application (www.ripseq.com). The RipSeq Mixed algorithm searches against the “16S human pathogen iSentio”—database currently containing about 1500 references. The definition of a positive identification with the RipSeq program has been described previously (9). Non-mixed chromatograms were analyzed with both RipSeq and a standard BLAST search against the GenBank database. For the BLAST searches interpretation criteria given by the CLSI were followed (11).

### Results:

### Validation of New Primers for PCR

The group-specific primers were aligned against 160 high quality GenBank references representing all 128 different genera present in the “16S human pathogen iSentio”—database.

The distribution of genera between the different primer pairs was: Primer pair A: 40 genera (Gram-positive cocci in chains (including anaerobes) and non-anaerobic Gram-positive rods including Mycobacteria); Primer pair B: 45 genera (Non-anaerobic Gram-negative bacteria); Primer pair C: 43 genera (Staphylococcus spp. and anaerobic bacteria except those covered for by primer A). A few exceptions to this genus distribution were seen, but all 128 genera examined were covered for by one of the primer pairs.

For each primer pair (A, B and C) cross-reactivity with the other bacterial groups were investigated by amplifying series of mixes with a falling ratio of target to non-target DNA (1:1-1:1000). In PCR reactions with primer pair A and primer pair C cloned 16S rDNA fragments were used. In reactions with primer pair B genomic DNA was used instead due to residual E. coli DNA from the cloning process. The highest tendency of cross-reactivity were between primer pair A (PCR “A”) and bacterial group C and between primer pair C (PCR “C”) and bacterial group A. For these combinations a 1000 fold higher copy number of non-target DNA was needed to reach equally high peaks as the target DNA in the resulting chromatograms. For primer pair B (PCR “B”) co-amplification of group “A” and “C” DNA was not detectable even when present at a 1000 fold higher concentration than group “B” target DNA. Cross-reactivity with human DNA was tested for each primer pair by amplifying 1000 pg, 100 pg, 10 pg, 1 pg and 0 pg of target bacterial DNA together with a constant amount of 100 ng human DNA (Promega). No cross-reactivity with human DNA was observed with any of the primer pairs. In the solutions with 100 ng of human DNA and no added bacterial DNA, only background bacterial DNA from the reagents was amplified.

### Samples

The poly-bacterial specimens contained high levels of bacterial DNA and with the universal PCR distances to the corresponding negative controls ranged from −20 to −8 cycles. For the specimens positive by PCR “A” the distance to the negative control ranged from −17 to −4 cycles. For the specimens negative by PCR “A” the distance ranged −2.4 to +3.9. The corresponding intervals for PCR “B” were −16 to −7 and −2.8 to +0.9 and for PCR “C” −17 to −6 and −2.0 to +4.4. (− indicates before the negative control and + indicates after).

Eighty percent of the samples were affected by antibiotics administrated prior to specimen collection and both standard direct 16S rRNA gene sequencing and direct sequencing using group-specific primers detected a higher number of bacteria than did culture. In total 37 species were found by culture, 51 by standard direct sequencing and 95 by the group-specific primers. A detailed comparison between these results is given in Table 5.

TABLE 5
Comparison of results obtained by culture, direct sequencing with a single
universal primer pair and direct sequencing with the group-specific primer pairs respectively.

| | Sample | | | | |
|---|---|---|---|---|---|
| ID | type | Culture | Universal primer | Group-specific primers | Antibiotic |
| 1 | Abscess | S. constellatus | Parvimonas sp.^{b} | F. nucleatum | CX, RI |
| | brain | | S. intermedius | G. haemolysans/morbillorum | |
| | | | | Parvimonas sp. | |
| | | | | S. intermedius | |
| 2 | Abscess | S. constellatus | S. intermedius | F. nucleatum | CX, RI |
| | brain | | | S. intermedius | |
| 3 | Abscess | Peptostreptococcus sp. | F. nucleatum^{b} | A. aphrophilus | CX, MZ |
| | brain | S. milleri group | P. micra | Eikenella sp. | |
| | | | S. intermedius | F. nucleatum | |
| | | | | F. nucleatum (polymorphum) | |
| | | | | N. elongata | |
| | | | | P. micra | |
| | | | | S. intermedius | |
| 4 | Abscess | No growth | F. nucleatum^{b} | A. meyeri | CT, MZ |
| | brain | | S. intermedius | C. gracilis | |
| | | | | F. nucleatum | |
| | | | | P. pleuritidis | |
| | | | | S. intermedius | |
| 5 | Abscess | A. meyeri | C. gracilis | A. meyeri | — |
| | brain | F. nucleatum | Fusobacterium sp. | C. gracilis | |
| | | | Peptostreptococcus sp. | Eikenella sp. | |
| | | | | Fusobacterium sp. | |
| | | | | Peptostreptococcus sp. | |
| 6 | Abscess | E. coli | E. coli/Shigella spp. | E. coli/Shigella spp. | AM |
| | liver | E. faecium | E. faecium/durans | E. faecium/durans | |
| | | Klebsiella sp. | | L. rhamnosus | |
| | | S. haemolyticus | | S. haemolyticus | |
| 7 | Abscess | S. haemolyticus | P. melaninogenica | G. haemolysans | PT |
| | liver | | S. mitis group | P. melaninogenica | |
| | | | | S. mitis group | |
| | | | | S. parasanguinis | |
| 8 | Abscess | No growth | C. perfringens | C. perfringens | CT |
| | liver^{a} | | E. coli/Shigella spp. | E. coli/Shigella spp. | |
| 9 | Abscess | S. milleri group | F. nucleatumb | E. corrodens | — |
| | muscle | | P. endodontalis | F. nucleatum | |
| | | | | P. micra | |
| | | | | P. circumdentaria | |
| | | | | P. endodontalis | |
| | | | | S. constellatus/intermedius | |
| 10 | Abscess | F. nucleatum | F. nucleatum | F. nucleatum | — |
| | ovarian^{a} | Peptostreptococcus sp. | Parvimonas sp. | Parvimonas sp. | |
| 11 | Abscess | B. fragilis | B. fragilis | B. fragilis | IP |
| | pancreas | E. faecium | | E. faecium | |
| 12 | Abscess | CNS | P. melaninogenica | A. meyeri | IP |
| | pancreas | | P. histicola | C. concisus | |
| | | | S. epidermidis | P. melaninogenica/histicola | |
| | | | | S. epidermidis | |
| 13 | Abscess | “Diphteroids” | C. tuberculostearicum | C. appendicis | — |
| | para-aortic | CNS | S. haemolyticus | C. tuberculostearicum | |
| | | | | S. haemolyticus | |
| 14 | Abscess | No growth | E. hormaechei | E. faecium | MP |
| | psoas | | | E. hormaechei | |
| 15 | Abscess | Enterococcus sp. | E. casseliflavus/ | E. casseliflavus/ | Unknown |
| | spleen | S. epidermidis | gallinarum^{b} | gallinarum | |
| | | | E. faecalis | E. coli | |
| | | | | E. faecalis | |
| | | | | F. nucleatum | |
| | | | | S. anginosus | |
| | | | | S. epidermidis | |
| 16 | Abscess | E. faecium | E. faecium | E. faecium | CI, OX |
| | Subcut.^{a} | F. magna | F. magna | F. magna | |
| 17 | Abscess | E. faecium | E. faecium/hirae | E. faecium/hirae | CI, MZ, |
| | Retro- | | K. pneumoniae | K. pneumoniae | LZ |
| | peritoneal^{a} | | | | |
| 18 | Bile^{a} | E. coli | E. coli/Shigella spp. | E. coli/Shigella spp. | CT, MZ |
| | | | F. nucleatum | F. nucleatum | |
| 19 | Bile | Enterococcus sp. | E. casseliflavus/ | E. caccae | IP |
| | | CNS | gallinarum | E. casseliflavus/gallinarum | |
| | | | S. epidermidis | E. faecalis | |
| | | | | S. epidermidis | |
| 20 | Empyema | No growth | P. micra^{b} | P. micra | PT |
| | | | P. stomatis | P. nigrescens | |
| | | | S. anginosus | P. stomatis | |
| | | | | S. anginosus | |
| 21 | Empyema^{a} | S. intermedius | C. gracilis | C. gracilis | CT, GE, |
| | | | Fusobacterium sp. | Fusobacterium sp. | PC |
| | | | S. intermedius | S. intermedius | |
| 22 | Empyema | S. intermedius | F. nucleatum | F. nucleatum | CT, PC |
| | | | P. micra | C. gracilis | |
| | | | S. intermedius | P. micra | |
| | | | | S. constellatus | |
| 23 | Empyema | S. constellatus | P. melaninogenica^{a} | C. gracilis | CI, CL |
| | | “Diphteroids” | | F. nucleatum | |
| | | CNS | | H. parainfluenzae | |
| | | | | P. melaninogenica | |
| | | | | P. micra | |
| | | | | S. constellatus | |
| 24 | Empyema | C. gracilis | P. pleuritidis | C. gracilis | PC |
| | | E. corrodens | | E. corrodens | |
| | | S. parasanguinis | | P. micra | |
| | | | | P. pleuritidis | |
| | | | | S. intermedius | |
| 25 | Empyema | Prevotella sp. | P. nigrescensb | P. nigrescens | CT |
| | | Ana. G-pos. rod | P. veroralis | P. veroralis | |
| | | | | D. invisus | |
| | | | | D. micraerophilus | |
| | | | | P. micra | |

^{a}Samples with identical results for standard direct sequencing and group-specific direct sequencing.
^{b}Chromatogram too complex to allow for complete analysis. Only dominant peaks included.
Antibiotics: — = No treatment, AM = Amoxicillin, CI = Ciprofloxacin, CL = Clindamycin, CT = Cefotaxime, CX = Ceftriaxone, GE = Gentamicin, IP = Imipenem, LZ = Linezolid, MP = Meropenem, MZ = Metronidazole, OX = oxacillin, PC = Penicillin-G, PT = Piperacillin- Tazobactam RI = Rifampicin, VA = Vancomycin
CNS: Coagulase negative Staphylococcus

All bacteria found with the standard universal primer pair were also detected with the group-specific primers. For six samples the two different sequencing protocols yielded identical results. In the remaining 19 samples a total of 44 additional bacteria were found by the group-specific primers ranging from 1 to 5 species per sample. Only 32 out of the 95 bacteria identified by group-specific direct sequencing were possible to cultivate. Four samples contained one or more isolates found exclusively by culture. These were a Staphylococcus haemolyticus isolated from sample 7, scarce growth of a coagulase negative Staphylococcus together with a “diphteroid” in sample 23 and two colonies of Streptococcus parasanguinis in sample 24. In addition a Klebsiella sp. isolated in sample 6 was not possible to retrieve from chromatogram 6B. Some of these isolates might represent sample contamination, but others are probably true findings that have not been detected by sequencing either because they had to compete for reagents with a more dominant species belonging to the same primer group or because direct sequencing can have lower sensitivity than culture in samples with living bacteria.

The detailed results from the group-specific PCRs for the poly-bacterial specimens are given in Table 6.

TABLE 6
Detailed results for Group-specific direct 16S sequencing from clinical samples.

| | Sample type | Primer pair A | Primer pair B | Primer pair C |
|---|---|---|---|---|
| 1 | Abscess brain | G. haemolysans/ | — | F. nucleatum |
| | | morbillorum | | |
| | | Parvimonas sp. | | |
| | | S. intermedius^{a} | | |
| 2 | Abscess brain | S. intermedius | — | F. nucleatum |
| 3 | Abscess brain | P. micra | A. aphrophilus^{b} | F. nucleatum |
| | | S. intermedius | E. corrodens | F. nucl. ssp. |
| | | | N. elongata | polymorphum |
| 4 | Abscess brain | A. meyeri | C. gracilis | F. nucleatum |
| | | S. intermedius | | P. pleuritidis |
| 5 | Abscess brain | A. meyeri | C. gracilis | Fusobacterium sp. |
| | | Peptostreptococcus sp. | Eikenella sp. | |
| 6 | Abscess liver | E. faecium/durans | E. coli/Shigella | L. rhamnosus |
| | | | Spp.^{b} | S. haemolyticus |
| 7 | Abscess liver | G. haemolysans | — | P. melaninogenica |
| | | S. mitis group | | (S. parasanguinis) |
| | | S. parasanguinis | | |
| 8 | Abscess liver | — | E. coli/Shigella | C. perfringens |
| | | | spp. | |
| 9 | Abscess mucle | P. micra | E. corrodens | F. nucleatum |
| | | S. constellatus/ | | P. circumdentaria |
| | | intermedius | | P. endodontalis |
| 10 | Abscess ovarian | Parvimonas sp. | — | F. nucleatum |
| 11 | Abscess pancreas | E. faecium | — | B. fragilis |
| | | (B. fragilis) | | |
| 12 | Abscess pancreas | A. meyeri | C. concisus | P. |
| | | (P. melaninogenica) | | melaninogenica/histicola |
| | | | | S. epidermidis |
| 13 | Abscess para- | C. tuberculostearicum | — | S. |
| | aortic | | | haemolyticus/epidermidis |
| | | | | C. appendicis |
| 14 | Abscess psoas | E. faecium | E. hormaechei | — |
| 15 | Abscess spleen | E. casseliflavus/ | E. coli | F. nucleatum |
| | | gallinarum | | |
| | | E. faecalis | | S. epidermidis |
| | | S. anginosus | | |
| 16 | Abscess | E. faecium | — | (E. faecium) |
| | subcutaneous | F. magna | | (F. magna) |
| 17 | Abscess | E. faecium/durans | K. pneumoniae | — |
| | retroperitoneal | | | |
| 18 | Bile | — | E. coli/Shigella | F. nucleatum |
| | | | spp. | |
| 19 | Bile | E. caccae | — | S. epidermidis |
| | | E. casseliflavus/ | | |
| | | gallinarum | | |
| | | E. faecalis | | |
| 20 | Empyema | P. micra | — | P. nigrescens |
| | | S. anginosus | | P. stomatis |
| 21 | Empyema | S. intermedius | C. gracilis | Fusobacterium sp. |
| 22 | Empyema | P. micra | C. gracilis | F. nucleatum |
| | | S. constellatus | | |
| 23 | Empyema | P. micra | C. gracilis | F. nucleatum |
| | | S. constellatus | H. parainfluenzae | P. melaninogenica |
| 24 | Empyema | P. micra | C. gracilis | P. pleuritidis |
| | | S. intermedius | E. corroders | |
| 25 | Empyema | D. invisus | — | P. nigrescens^{b} |
| | | D. micraerophilus | | P. veroralis |
| | | P. micra | | |

^{a}Differentiation between S. constellatus and S. intermedius based on type strains only.
^{b}Chromatogram too complex to allow for complete analysis. Only dominant peaks included.
Species in parenthesis (n = 5) indicates cross reactivity between primer groups.

Four cases of cross-reactivity between primer groups were observed. In sample 7, a Group A species was detected as low secondary peaks in chromatogram 7C. In both samples 11 and 12 a species belonging to Group C was also detectable as the lower peaks in the A chromatograms. In sample 16, Enterococcus faecium and Finegoldia magna were amplified by both PCR “A” and “C”, but PCR “A” became positive 8,6 cycles prior to PCR “C”. Three of the group-specific chromatograms were found to be too complex to be completely resolved, indicating that these samples still might contain one or more unidentifiable species.

Among the 25 clinical specimens assumed to be negative (Table 7) one sample (ID 35) was found to be positive by the universal PCR. It reached Ct at cycle 31,5; exactly three cycles before the negative control. Melt point analysis showed no distinct peak and it was negative by the group-specific PCRs. Sequencing was unsuccessful and the PCR result was defined as a false positive. Another sample (ID 50) was positive by PCR “B”. This reached Ct at cycle 30.6; 3.1 cycles before the closest negative control. Melt point analysis gave an irregular peak. Sequencing produced a complex chromatogram dominated by a Pseudomonas and an Acidovorax sp. This corresponds to what we typically find in our negative controls and the PCR-reaction was judged to be a false positive. The remaining 98 PCR reactions were negative as expected.

TABLE 7
Presentation of the presumably sterile samples including the results for
culture and the two direct sequencing protocols.

| | | | | Group Specific | | | |
|---|---|---|---|---|---|---|---|
| | | | Universal | PCRs | | | |
| ID | Specimen | Culture | PCR | A | B | C | Antibiotics |
| 26 | Abscess liver (amoebic) | No growth | Neg | Neg | Neg | Neg | Unknown |
| 27 | Abscess lung (aspergilloma) | No growth | Neg | Neg | Neg | Neg | CT |
| 28 | Abscess neck (Tumor colli) | No growth | Neg | Neg | Neg | Neg | — |
| 29 | Abscess pelvic (Cancer) | No growth | Neg | Neg | Neg | Neg | Unknown |
| 30 | Abscess subcutaneous (Cancer) | No growth | Neg | Neg | Neg | Neg | CF |
| 31 | Biopsy bone | No growth | Neg | Neg | Neg | Neg | — |
| 32 | Pericardial fluid | No growth | Neg | Neg | Neg | Neg | GE, PC |
| 33 | Pericardial fluid | No growth | Neg | Neg | Neg | Neg | AM, CI |
| 34 | Pericardial fluid | No growth | Neg | Neg | Neg | Neg | Unknown |
| 35 | Peritoneal fluid | No growth | Pos^{a} | Neg | Neg | Neg | OX |
| 36 | Peritoneal fluid | No growth | Neg | Neg | Neg | Neg | — |
| 37 | Pleural fluid | No growth | Neg | Neg | Neg | Neg | — |
| 38 | Pleural fluid | No growth | Neg | Neg | Neg | Neg | — |
| 39 | Pleural fluid | No growth | Neg | Neg | Neg | Neg | — |
| 40 | Pleural fluid | No growth | Neg | Neg | Neg | Neg | — |
| 41 | Pleural fluid | No growth | Neg | Neg | Neg | Neg | — |
| 42 | Pleural fluid | No growth | Neg | Neg | Neg | Neg | Unknown |
| 43 | Subdural hematoma | No growth | Neg | Neg | Neg | Neg | — |
| 44 | Synovial fluid | No growth | Neg | Neg | Neg | Neg | Unknown |
| 45 | Synovial fluid | No growth | Neg | Neg | Neg | Neg | — |
| 46 | Synovial fluid | No growth | Neg | Neg | Neg | Neg | Unknown |
| 47 | Synovial fluid | No growth | Neg | Neg | Neg | Neg | — |
| 48 | Tissue prosthetic joint | No growth | Neg | Neg | Neg | Neg | Unknown |
| 49 | Tissue prosthetic joint | No growth | Neg | Neg | Neg | Neg | — |
| 50 | Tissue prosthetic joint | No growth | Neg | Neg | Pos^{b} | Neg | — |

^{a}Distance to negative ctrl. = 3.0 cycles; Ct = 31.5.
^{b}Distance to closest negative ctrl = 3.1 cycles; Ct = 30.6
AM = amoxicillin,
CI = ciprofloxacin,
CF = cefalotin,
CT = cefotaxime,
GE = gentamicin,
OX = oxacillin,
PC = penicillin

### Discussion:

Detection and identification of bacteria directly from poly-microbial clinical samples by broad-range PCR followed by DNA sequencing can be accomplished using RipSeq mixed chromatogram analysis. In this study we replaced the standard universal 16S rRNA PCR with a set of three separate group-specific PCRs reducing the risk for species present at lower concentrations to become outcompeted in the amplification step. The importance of this factor is illustrated by the fact that only eight of the chromatograms obtained with the universal primers were judged to contain more than three bacterial sequences (Table 5).

The group-specific PCRs significantly increased the number of identified bacteria as compared to amplification with a universal PCR (95 versus 51, p<0.05 by Wilcoxon signed-rank test). All bacteria identified by standard universal direct sequencing and RipSeq analysis were also found with the group-specific primers and RipSeq analysis. Since the species combinations in the chromatograms obtained with the universal versus the group-specific primers for the most part were different, this is a confirmation of RipSeq's ability to analyse mixed DNA chromatograms. In addition 18 of the bacteria identified as part of a mixed chromatogram with the universal primer pair were recovered as pure chromatograms with one of the group-specific primer pairs.

In a set of group-specific primers the number of primer pairs will be a compromise between the possibilities given by the chosen gene target, sensitivity and practical considerations. It was found to be advantageous that the same cycling conditions could be used for all PCRs and that a single primer could be used for all cycle sequencing reactions. For maximum effect, relevant bacterial species should distribute evenly between the primer pairs and cross-reactivity should be low. We found an almost perfect distribution for the species included in the pre-study alignments, and we also saw a good distribution in the study; 42 species belonged to Primer pair A, 20 to Primer pair B and 33 to Primer pair C. Given that two targets belong to different primer groups the primers described here will handle ratios of 1:1000 or higher. With two targets from the same group the situation will be equivalent to what we see with a single universal primer pair were successful detection of both targets cannot be expected with ratios higher than 1:10.

A negative control containing a mixed DNA population in low concentration could give low reproducibility with the group-specific primers due to random distribution of the different DNA targets in the respective reaction tubes. We also considered that low level DNA contamination in a specimen could have a different composition than low level DNA contamination in the negative control (due to e.g. sample collection and transportation tubes). Consequently, in reactions where the negative control did not contain any target DNA a poor amplification would lead to a very high Ct value that subsequently could define a negative sample as positive. For these reasons, although the negative control was included in all group specific PCRs; the PCR reaction in which it became positive first defined the cut-off for a positive sample for all three reactions. With the definition of a positive PCR used in this study, we got two false positive PCR-reactions. We therefore suggest including a second criterion stating that a sample in addition to becoming positive 3 cycles before the negative control has to reach Ct before cycle 30 in order to be defined as positive. In our experience reactions with higher Ct values are of doubtful significance and rarely result in relevant findings.

By using group-specific primers the cost per analysis will see some increase. We kept the increase in both price and workload as low as possible by sequencing in one direction only and the average number of sequencing reactions in our material was 2.55 per positive sample. Several publications based on mono-directional sequencing exist, and it is also used in a commercially available 16S sequencing kit (3, 4, 15). With the high fidelity of today's PCR and Sanger sequencing reactions the main reason for bi-directional sequencing is to obtain maximum read lengths and better discrimination between similar species. If we ignore the primer binding areas which are of no value for identification, a reduction of 40-50 readable bases can be expected with mono-directional versus bi-directional sequencing. With our primers this lead to a 40-50% reduction in the number of readable bases in the variable area V3, whereas the variable areas V1 and V2 remained intact (2). We find the potential reduction in resolution to the species level defendable when compared to the significant increase in identifiable bacteria. The only visible consequence of mono-directional sequencing in this study was found in sample 12 where the better discrimination between P. histicola and P. melaninogenica in the forward direction allowed for an “and” instead of an “and/or” identification with the universal protocol. A rational approach will be to use a single universal primer pair for specimens expected to be mono-bacterial (e.g. CSF and synovial fluids) and reserve the group-specific primers for specimens known to frequently contain multiple organisms (e.g. abscesses, pleural fluids and bile).

It is clear from this study and other studies that the results achieved by routine culture-based diagnostics alone is not sufficient when it comes to patients who have received one or more doses of antibiotics prior to sample collection or in the investigation of anaerobe infections (1, 7, 10, 12-14). Infectious disease physicians are well aware of these limitations. As a consequence microbiological results are currently not so often used to tailor individual antibiotic treatment, but more so to look for the unexpected organisms that eventually should lead to a diversion from standard empiric therapy. Compared to culture, sequencing with group-specific primers detected on average 2.5 times as many species per sample and although the unexpected was not found in this study a stronger assurance of adequate therapy is in itself of great value for the physician in charge. In situations where first line empiric treatment cannot be used or when patients are to be transferred to oral treatment a complete bacteriological result is of even greater importance.

The purposes of this study were to demonstrate the potential of the Group-specific PCRs and to establish a robust protocol. The approach has the advantage that it can be readily implemented in any diagnostic laboratory with experience in standard direct sequencing.

### REFERENCES

1. Al Masalma, M., F. Armougom, W. M. Scheld, H. Dufour, P. H. Roche, M. Drancourt, and D. Raoult. 2009. The expansion of the microbiological spectrum of brain abscesses with use of multiple 16S ribosomal DNA sequencing. Clin. Infect. Dis. 48:1169-1178. doi: 10.1086/597578.
2. Baker, G. C., J. J. Smith, and D. A. Cowan. 2003. Review and re-analysis of domain-specific 16S primers. J. Microbiol. Methods. 55:541. doi: 10.1016/j.mimet.2003.08.009.
3. Bosshard, P. P., S. Abels, M. Altwegg, E. C. Bottger, and R. Zbinden. 2004. Comparison of conventional and molecular methods for identification of aerobic catalase-negative gram-positive cocci in the clinical laboratory J. Clin. Microbiol. 42:2065-2073.
4. Bosshard, P. P., S. Abels, R. Zbinden, E. C. Bottger, and M. Altwegg. 2003. Ribosomal DNA sequencing for identification of aerobic gram-positive rods in the clinical laboratory (an 18-month evaluation) J. Clin. Microbiol. 41:4134-4140.
5. Bosshard, P. P., R. Zbinden, and M. Altwegg. 2002. Turicibacter sanguinis gen. nov., sp. nov., a novel anaerobic, Gram-positive bacterium. Int. J. Syst. Evol. Microbiol. 52:1263-1266.
6. Edwards, U., T. Rogall, H. Blocker, M. Emde, and E. C. Bottger. 1989. Isolation and direct complete nucleotide determination of entire genes. Characterization of a gene coding for 16S ribosomal RNA. Nucleic Acids Res. 17:7843-7853.
7. Goldenberger, D., A. Kunzli, P. Vogt, R. Zbinden, and M. Altwegg. 1997. Molecular diagnosis of bacterial endocarditis by broad-range PCR amplification and direct sequencing J. Clin. Microbiol. 35:2733-2739.
8. Harris, K. A., K. J. Fidler, J. C. Hartley, J. Vogt, N. J. Klein, F. Monsell, and V. M. Novelli. 2002. Unique case of Helicobacter sp. osteomyelitis in an immunocompetent child diagnosed by broad-range 16S PCR. J. Clin. Microbiol. 40:3100-3103.
9. Kommedal, O., B. Karlsen, and O. Saebo. 2008. Analysis of mixed sequencing chromatograms and its application in direct 16S rRNA gene sequencing of polymicrobial samples. J. Clin. Microbiol. 46:3766-3771. doi: 10.1128/JCM.00213-08.
10. Kommedal, O., K. Kvello, R. Skjastad, N. Langeland, and H. G. Wiker. 2009. Direct 16S rRNA gene sequencing from clinical specimens, with special focus on polybacterial samples and interpretation of mixed DNA chromatograms J. Clin. Microbiol. 47:3562-3568. doi: 10.1128/JCM.00973-09.
11. Petti, C. A., P. P. Bosshard, M. E. Brandt, J. E. Clarridge III, T. V. Feldblyum, P. Foxall, M. R. Furtado, N. Pace, and G. Procop. 2008. Interpretive Criteria for Identification of Bacteria and Fungi by DNA Target Sequencing; Approved Guideline. MM18-A:.
12. Petti, C. A., K. E. Simmon, J. Bender, A. Blaschke, K. A. Webster, M. F. Conneely, P. C. Schreckenberger, T. C. Origitano, and M. Challapalli. 2008. Culture-Negative intracerebral abscesses in children and adolescents from Streptococcus anginosus group infection: a case series. Clin. Infect. Dis. 46:1578-1580. doi: 10.1086/587655.
13. Schuurman, T., R. F. de Boer, A. M. Kooistra-Smid, and A. A. van Zwet. 2004. Prospective study of use of PCR amplification and sequencing of 16S ribosomal DNA from cerebrospinal fluid for diagnosis of bacterial meningitis in a clinical setting. J. Clin. Microbiol. 42:734-740.
14. Senn, L., M. Franciolli, D. Raoult, A. Moulin, L. Von Segesser, T. Calandra, and G. Greub. 2005. Coxiella burnetii vascular graft infection. BMC Infect. Dis. 5:109. doi: 10.1186/1471-2334-5-109.
15. Wellinghausen, N., A. J. Kochem, C. Disque, H. Muhl, S. Gebert, J. Winter, J. Matten, and S. G. Sakka. 2009. Diagnosis of bacteremia in whole-blood samples by use of a commercial universal 16S rRNA gene-based PCR and sequence analysis J. Clin. Microbiol. 47:2759-2765. doi: 10.1128/JCM.00567-09.

1. An oligonucleotide primer set for direct 16S rDNA sequencing of a polybacterial sample, comprising at least two oligonucleotide primers of formula:
5′ xz 3′
wherein x is sequence of nucleotides that hybridises to the complement of the region of the 16S rRNA gene of Escherichia coli from position 9 to position 27 and z is at least one nucleotide that provides specificity to a group of bacteria.

2. The oligonucleotide primer set according to claim 1, wherein z is a dinucleotide, a trinucleotide or a tetranucleotide.

3. The oligonucleotide primer set according to claim 2, consisting of:
(a) an oligonucleotide primer comprising the sequence x₁gAc or x₁rAc;
(b) an oligonucleotide primer comprising the sequence x₂Akt, x₂gAk, x₂aKt or x₂aKtg; and
(c) an oligonucleotide primer comprising the sequence x₃gAt;
wherein x₁, x₂ and x₃ are each sequences of nucleotides which hybridize to the complement of the region of the 16S rRNA gene of Escherichia coli from position 9 to position 27, and wherein a capital letter indicates a locked nucleic acid (LNA).

4. The oligonucleotide primer set according to claim 3, wherein one or more of x₁, x₂ and x₃ hybridize to the complement of the region of the 16S rRNA gene of Escherichia coli from position 9 to position 27 under stringent conditions.

5. The oligonucleotide primer set according to claim 3 consisting of:
(a) an oligonucleotide primer consisting of the nucleotide sequence of any one of SEQ ID Nos. 3 to 5;
(b) an oligonucleotide primer consisting of the nucleotide sequence of any one of SEQ ID Nos. 6 to 13; and
(c) an oligonucleotide primer consisting of the nucleotide sequence of any one of SEQ ID Nos. 14 to 16.

6. The oligonucleotide primer set according to claim 3 consisting of:
(a) an oligonucleotide primer consisting of the nucleotide sequence of SEQ ID No. 4;
(b) an oligonucleotide primer consisting of the nucleotide sequence of SEQ ID No. 9; and
(c) an oligonucleotide primer consisting of the nucleotide sequence of SEQ ID No. 14.

7. A kit for direct 16S rDNA sequencing of a polybacterial sample, comprising at least two oligonucleotide primers of formula:
5′ xz 3′
where x is a sequence of nucleotides that hybridises to the complement of the region of the 16S rRNA gene of Escherichia coli from position 9 to position 27 and z is at least one nucleotide that provides specificity to a group of bacteria.

8. The kit according to claim 7, wherein z is a dinucleotide, a trinucleotide or a tetranucleotide.

9. The kit according to claim 7, comprising:
(a) an oligonucleotide primer comprising the sequence x₁gAc or x₁rAc;
(b) an oligonucleotide primer comprising the sequence x₂Akt, x₂gAk, x₂aKt or x₂aKtg; and
(c) an oligonucleotide primer comprising the sequence x₃gAt;
wherein x₁, x₂ and x₃ are each sequences of nucleotides which hybridize to the complement of the region of the 16S rRNA gene of Escherichia coli from position 9 to position 27, and wherein a capital letter indicates a locked nucleic acid (LNA).

10. The kit according to claim 9, wherein one or more of x₁, x₂ and x₃ hybridize to the complement of the region of the 16S rRNA gene of Escherichia coli from position 9 to position 27 under stringent conditions.

11. The kit according to claim 9, comprising:
(a) an oligonucleotide primer consisting of the nucleotide sequence of any one of SEQ ID Nos. 3 to 5;
(b) an oligonucleotide primer consisting of the nucleotide sequence of any one of SEQ ID Nos. 6 to 13; and
(c) an oligonucleotide primer consisting of the nucleotide sequence of any one of SEQ ID Nos. 14 to 16.

12. The kit according to claim 9, comprising:
(a) an oligonucleotide primer consisting of the nucleotide sequence of SEQ ID No. 4;
(b) an oligonucleotide primer consisting of the nucleotide sequence of SEQ ID No. 9; and
(c) an oligonucleotide primer consisting of the nucleotide sequence of SEQ ID No. 14.

13. The kit according to claim 7, further comprising a reverse primer consisting of the sequence of SEQ ID No. 17.

14. A method of direct sequencing of 16S rDNA in a polybacterial sample, the method comprising the steps of:
(a) splitting the sample into at least two aliquots;
(b) conducting a first PCR amplification with a first aliquot using a first forward primer of formula 5′ xz 3′ and a suitable reverse primer;
(c) conducting a second PCR amplification with a second aliquot using a second forward primer of formula 5′ xz 3′ which is of different sequence to the first forward primer and a suitable reverse primer; and
(d) sequencing the PCR products obtained in steps (b) to (c) using suitable sequencing primers;
wherein x is an oligonucleotide which hybridizes to the complement of the region of the 16S rRNA gene of Escherichia coli from position 9 to position 27, and wherein z is at least one nucleotide that provides specificity to a group of bacteria, wherein the first and second forward primers are specific for different groups of bacteria, and wherein steps (b) to (c) may be carried out concurrently or consecutively in any order.

15. The method according to claim 14, wherein z is a dinucleotide, a trinucleotide or a tetranucleotide.

16. The method according to claim 14, comprising the steps of:
(a) splitting the sample into at least three aliquots;
(b) conducting a PCR amplification with a first aliquot using forward primers comprising the sequence x₁gAc or x₁rAc and a suitable reverse primer;
(c) conducting a PCR amplification with a second aliquot using forward primers comprising the sequence x₂Akt, x₂gAk, x₂aKt or x₂aKtg and a suitable reverse primer;
(d) conducting a PCR amplification with a third aliquot using forward primers comprising the sequence x₃gAt and a suitable reverse primer; and
(e) sequencing the PCR products obtained in steps (b) to (d) using suitable sequencing primers;
wherein x₁, x₂ and x₃ are each nucleotide sequences which hybridize to the complement of the region of the 16S rRNA gene of Escherichia coli from position 9 to position 27, and wherein a capital letter indicates a locked nucleic acid (LNA), and wherein steps (b) to (d) may be carried out concurrently or consecutively in any order.

17. The method according to claim 16, wherein one or more of x₁, x₂ and x₃ hybridize to the complement of the region of the 16S rRNA gene of Escherichia coli from position 9 to position 27 under stringent conditions.

18. The method according to claim 16, wherein the primers for steps (b) to (d) are selected from:
(a) a primer consisting of the nucleotide sequence of any one of SEQ ID Nos. 3 to 5;
(b) a primer consisting of the nucleotide sequence of any one of SEQ ID Nos. 6 to 13; and
(c) a primer consisting of the nucleotide sequence of any one of SEQ ID Nos. 14 to 16.

19. The method according to claim 16, wherein:
(a) a primer consisting of the nucleotide sequence of SEQ ID No. 4;
(b) a primer consisting of the nucleotide sequence of SEQ ID No. 9; and
(c) a primer consisting of the nucleotide sequence of SEQ ID No. 14.

20. The method according to claim 14, wherein the sequencing primers correspond to the forward PCR primers used to generate the PCR product being sequenced.

21. The method according to claim 14, wherein the sample comprises river water, pond water, lake water, sea water, waste water, abscesses in internal organs (brain, lung, spleen, liver, pancreas, kidney, ovaries, aorta), deep soft-tissue or muscular abscesses, retroperitoneal abscesses, aspirate/biopsies from spondylodiscitis and other bone-related infections, pus, pleural fluids, blood, bile, urine or saliva.
